(19) United States
(12) Patent Application Publication
(10) Pub. No.: US 2022/0225914 A1
(43) Pub. Date: Jul. 21, 2022

(54) Guidewire Delivery Device and Related Devices, Systems and Methods

(71) Applicant: Becton, Dickinson and Company, Franklin Lakes, NJ (US)

(72) Inventors: Xiwei Chen, Suzhou (CN); Bo Yan, Shanghai (CN); Yueqiang Xue, Shanghai (CN)

(21) Appl. No.: 17/579,079

(22) Filed: Jan. 19, 2022

Related U.S. Application Data
(60) Provisional application No. 63/139,656, filed on Jan. 20, 2021.

(51) Int. Cl. A61B 5/15; A61M 25/09

(52) U.S. Cl. CPC A61B 5/150992; A61M 2025/0681; A61M 25/09

(57) ABSTRACT

A guidewire delivery device may include a housing, which may include a distal end, a proximal end, a lumen extending through the distal end of the housing and the proximal end of the housing, and a slot disposed between the distal end of the housing and the proximal end of the housing. The tab may be moveable with respect to the slot. The guidewire delivery device may include a penetration cannula coupled to the tab and extending in a distal direction. The guidewire delivery device may include an adapter proximal to and in fluid communication with the penetration cannula.

### CROSS-REFERENCE TO RELATED APPLICATION

The present application claims priority to U.S. Provisional Application Ser. No. 63/139,656, entitled “Guidewire Delivery Device and Related Devices, Systems and Methods”, filed Jan. 20, 2021, the entire disclosure of which is hereby incorporated by reference in its entirety.

### BACKGROUND

A catheter is commonly used to infuse fluids into vasculature of a patient. For example, the catheter may be used for infusing normal saline solution, various medicaments, or total parenteral nutrition.

The catheter may include a peripheral intravenous (“IV”) catheter. In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may remove the introducer needle, leaving the catheter in place for future fluid infusion.

Overtime, thrombosis may accumulate at the catheter, and the catheter may narrow, collapse, or clog, leading to failure of the catheter. The thrombosis makes it difficult to draw blood through the catheter that is indwelling. In response to patency of the catheter being compromised, the catheter may need to be removed from the patient. The catheter may then be replaced with another catheter, which is usually introduced via another needle stick, leading to discomfort for the patient. Moreover, typical methods of blood collection through the catheter that is indwelling often result in insufficient blood volume and blood coagulation.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure generally relates to a blood collection device and related devices, systems, and methods. In particular, the present disclosure relates to a guidewire delivery device and related devices, systems, and methods. In some embodiments, the guidewire delivery device may include a housing, which may include a distal end, a proximal end, and a lumen extending through the distal end of the housing and the proximal end of the housing. In some embodiments, the housing may include a slot disposed between the distal end of the housing and the proximal end of the housing.

In some embodiments, the guidewire delivery device may include a tab moveable with respect to the slot. In some embodiments, the tab may extend through the slot and/or may be movable along the slot. In some embodiments, the guidewire delivery device may include a penetration cannula coupled to the tab and extending in a distal direction. In some embodiments, the guidewire delivery device may include an adapter proximal to and in fluid communication with the penetration cannula. In some embodiments, the adapter may include a Y-adapter or another suitable adapter. In some embodiments, the penetration cannula and the adapter may be configured to receive a guidewire therethrough.

In some embodiments, the guidewire delivery device may include a lumen extending from a proximal end of the penetration cannula to the adapter. In some embodiments, the lumen may extend through the proximal end of the housing. In some embodiments, in response to movement of the tab with respect to the slot in a distal direction, the adapter may be closer to the proximal end of the housing. In some embodiments, the guidewire delivery device may include a body and an extension tube extending proximally from the body to the adapter. In some embodiments, the tab may extend from the body. In some embodiments, the penetration cannula may extend distally from the body.

In some embodiments, the distal end of the housing may include a luer adapter configured to couple to a side port or a distal end of a catheter adapter. In some embodiments, the penetration cannula may be 20 G or another suitable gauge. In some embodiments, the guidewire delivery device may include the guidewire extending distally through the adapter.

In some embodiments, the guidewire may be coiled. In some embodiments, a distal end of the guidewire may include multiple elongated arms. In some embodiments, the elongated arms may be configured to separate in response to removal of an inward biasing force.

In some embodiments, a method of blood collection may include coupling the guidewire delivery device to a catheter assembly. In some embodiments, the catheter assembly may include a catheter adapter, which may include a distal end, a proximal end, and a lumen extending through the distal end of the catheter adapter and the proximal end of the catheter adapter. In some embodiments, the catheter assembly may include a catheter extending distally from the distal end of the catheter adapter. In some embodiments, the catheter assembly may include a septum configured to seal a fluid pathway through the catheter assembly.

In some embodiments, the method may include advancing the tab distally within the slot. In some embodiments, in response to advancing the tab distally, the penetration cannula may penetrate the septum. In some embodiments, after advancing the tab distally with respect to the slot, the method may include advancing the guidewire distally through the penetration cannula and/or distal to the catheter. In some embodiments, the septum may be disposed within the lumen of the catheter adapter. In some embodiments, coupling the guidewire delivery device to the catheter assembly may include coupling the proximal end of the housing to the proximal end of the catheter adapter.

In some embodiments, the catheter adapter may include the side port, which may be disposed between the distal end of the catheter adapter and the proximal end of the catheter adapter. In some embodiments, the catheter assembly may include another adapter. In some embodiments, the catheter assembly may include an extension tube, which may include a distal end coupled to the side port and a proximal end coupled to the other adapter. In some embodiments, the septum may be disposed within the other adapter.

In some embodiments, coupling the guidewire delivery device to the catheter assembly may include coupling the proximal end of the housing to the other adapter. In some embodiments, the catheter assembly may include a wedge disposed within the lumen of the catheter adapter. In some embodiments, the catheter may be secured within the distal end of the catheter adapter by the wedge. In some embodiments, in response to advancing the tab distally with respect to the slot, a distal end of the penetration cannula may be disposed within the wedge.

In some embodiments, the distal end of the guidewire may include the elongated arms, and the elongated arms may be biased together in response to the elongated arms being in the catheter. In some embodiments, the elongated arms are configured to separate in response to the elongated arms moving distal to the catheter, wherein after the tab is advanced distally with respect to the slot, the guidewire is advanced distally through the penetration cannula and distal to the catheter and rotated.

In some embodiments, the adapter may include a distal port, a first proximal port, and a second proximal port. In some embodiments, a needleless connector may be coupled to the first proximal port. In some embodiments, a blood collection device is coupled to the second proximal port. In some embodiments, after advancing the tab distally with respect to the slot, the guidewire may be advanced distally through the first proximal port.

In some embodiments, after advancing the tab distally with respect the slot, the guidewire may be advanced distally through the penetration cannula and distal to the catheter. In some embodiments, the method may include retracting the guidewire proximally such that a distal end of the guidewire is disposed within the penetration cannula. In some embodiments, after retracting the guidewire proximally such that the distal end of the guidewire is disposed within the penetration cannula, the method may include collect blood through a blood collection device coupled to the adapter. In some embodiments, the method may include retracting the tab proximally with respect to the slot. In some embodiments, in response to retracting the tab proximally with respect to the slot, the penetration cannula is withdrawn proximally through the septum.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is a cross-sectional view of an example guidewire delivery device, according to some embodiments;

FIG. 1B is an upper perspective view of the guidewire delivery device with an example housing removed for illustrative purposes, according to some embodiments;

FIG. 2A is an upper perspective view of an example catheter system, illustrating the guidewire delivery device coupled to a proximal end of an example catheter adapter, according to some embodiments;

FIG. 2B is a cross-sectional view of the catheter system of FIG. 2A, illustrating the guidewire delivery device coupled to the proximal end of the catheter adapter, according to some embodiments;

FIG. 2C is an enlarged view of a distal end of the catheter system of FIG. 2A, illustrating an example guidewire in an example advanced position, according to some embodiments;

FIG. 3A is an upper perspective view of an example catheter system, illustrating the guidewire delivery device coupled to a proximal port of another example adapter, according to some embodiments;

FIG. 3B is an upper perspective view of a distal portion of the catheter system of FIG. 3A, illustrating the guidewire in the advanced position, according to some embodiments;

FIG. 3C is an enlarged view of a distal end of the catheter system of FIG. 3A, illustrating the guidewire in the advanced position, according to some embodiments;

FIG. 3D is a cross-sectional view of a portion of the catheter system of FIG. 3A, according to some embodiments;

FIG. 4A is an upper perspective view of the catheter system of FIG. 2A, illustrating an example catheter assembly ready for insertion into vasculature of a patient, according to some embodiments;

FIG. 4B is an upper perspective view of the catheter system of FIG. 2A, illustrating an example tab in an example advanced position, according to some embodiments;

FIG. 4C is an upper perspective view of the catheter system of FIG. 2A, illustrating the guidewire in the advanced position and the catheter system ready for blood draw, according to some embodiments;

FIG. 4D is an upper perspective view of the catheter system of FIG. 3A, illustrating the guidewire in the advanced position and the catheter system ready for blood draw, according to some embodiments;

FIG. 5A is an upper perspective view of a distal end of an example guidewire disposed in the advanced position within an example catheter, according to some embodiments;

FIG. 5B is an upper perspective view of the catheter system of FIG. 2A, illustrating the catheter assembly ready for insertion into the vasculature of the patient, according to some embodiments;

FIG. 5C is an upper perspective view of the catheter system of FIG. 2A, illustrating the tab in the advanced position, according to some embodiments;

FIG. 5D is an upper perspective view of the catheter system of FIG. 2A, illustrating the guidewire of FIG. 5A in the advanced position and the catheter system ready for blood draw, according to some embodiments; and

FIG. 5E is an upper perspective view of the catheter system of FIG. 2A, illustrating the guidewire of FIG. 5A in a retracted position, according to some embodiments.

### DESCRIPTION OF EMBODIMENTS

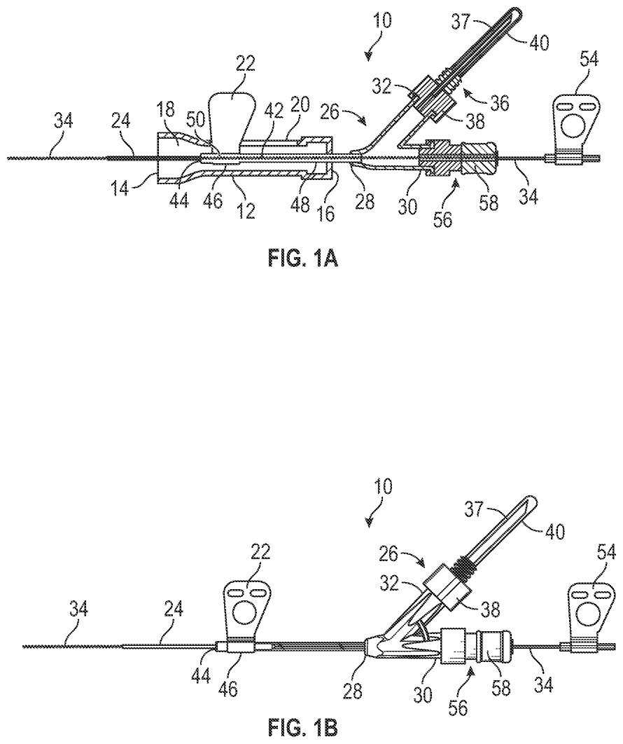

Referring now to FIGS. 1A-1B, a guidewire delivery device 10 is illustrated, according to some embodiments. In some embodiments, the guidewire delivery device 10 may include a housing 12, which may include a distal end 14, a proximal end 16, and a lumen 18 extending through the distal end 14 of the housing 12 and the proximal end 16 of the housing 12. In some embodiments, the housing 12 may include a slot 20 disposed between the distal end 14 of the housing 12 and the proximal end 16 of the housing 12.

In some embodiments, the guidewire delivery device 10 may include a tab 22 moveable with respect to or along the slot 20. In some embodiments, the tab 22 may extend through the slot 20. In some embodiments, the guidewire delivery device 10 may include a penetration cannula 24 extending in a distal direction. In some embodiments, the penetration cannula 24 may be coupled to the tab 22 such that the penetration cannula 24 moves along with the tab 22.

In some embodiments, the guidewire delivery device 10 may include an adapter 26 proximal to and in fluid communication with the penetration cannula 24. In some embodiments, the adapter 26 may include a Y-adapter or another suitable adapter. In some embodiments, the adapter 26 may include a distal port 28, a first proximal port 30, and a second proximal port 32. In some embodiments, the penetration cannula 24 and the adapter 26 may be configured to receive a guidewire 34 therethrough. In some embodiments, after advancing the tab 22 distally with respect to the slot 20, the guidewire 34 may be inserted and/or advanced distally through the first proximal port 30.

FIG. 1A illustrates the tab 22 in an advanced position, according to some embodiments. In some embodiments, in response to the tab 22 being in the advanced position, the tab 22 may be disposed at a distal end of the slot 20, which may act as a stop to prevent further movement of the tab 22 in the distal direction.

In some embodiments, a blood collection device may be coupled to the second proximal port 32. In some embodiments, the blood collection device may be coupled to the second proximal port 32 via a blood collection adapter 36, which may be directly coupled to the second proximal port 32. In some embodiments, the blood collection device may include a blood collection tube or the BD VACUTAINER® Blood Collection Tube, available from Becton Dickinson & Company. In some embodiments, the blood collection adapter 36 may include a needle 37 extending from a luer adapter 38. In some embodiments, an elastomeric sheath 40 may cover the needle 37. In some embodiments, in response to coupling the blood collection device to the blood collection adapter 36, a sharp tip of the needle 37 may pierce the elastomeric sheath 40, the elastomeric sheath 40 may be compressed towards the luer adapter 38 and the needle 37 may be inserted into the blood collection device.

In some embodiments, the guidewire delivery device 10 may include a lumen 42 extending from a proximal end 44 of the penetration cannula 24 to the adapter 26, which may include another lumen. In some embodiments, the lumen 42 may extend through the proximal end 16 of the housing 12. In some embodiments, in response to movement of the tab 22 with respect to the slot 20 in the distal direction, the adapter 26 may be closer to the proximal end 16 of the housing 12. In some embodiments, the penetration cannula 24 may be 20 G or another suitable gauge. In some embodiments, the penetration cannula 24 may include a tube.

In some embodiments, the guidewire delivery device 10 may include a body 46 and an extension tube 48 extending proximally from the body 46 to the adapter 26. In some embodiments, the tab 22 may extend from the body 46. In some embodiments, the penetration cannula 24 may extend distally from the body 46. In some embodiments, a fluid pathway 50 of the guidewire delivery device 10 may extend through one or more of following: the penetration cannula 24, the body 46, the extension tube 48, and the adapter 26. In some embodiments, the lumen 42 may extend through the body 46 and/or the extension tube 48. In some embodiments, the fluid pathway 50 may extend through the lumen 42.

In some embodiments, the distal end 14 of the housing 12 may include a luer adapter 52 configured to couple to a side port or a distal end of a catheter adapter. In some embodiments, the guidewire delivery device 10 may include the guidewire 34 extending distally through the adapter 26. In some embodiments, a proximal end of the guidewire 34 may be secured within another tab 54. In some embodiments, a clinician may pinch the tab 22 to advance or retract the penetration cannula 24. In some embodiments, the clinician may pinch the other tab 54 to advance and/or withdraw the guidewire 34.

In some embodiments, a needleless connector 56 may be coupled to the first proximal port 30. In some embodiments, the needleless connector 56 may include a septum 58. In some embodiments, the guidewire 34 may be inserted through the needleless connector 56.

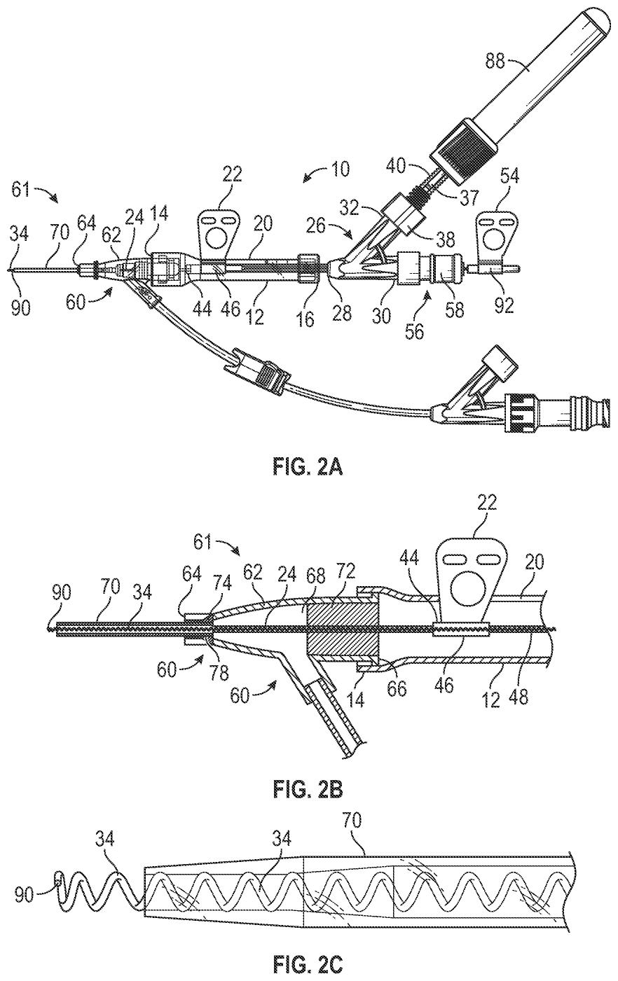

Referring now to FIGS. 2A-2B, in some embodiments, the guidewire delivery device 10 may be coupled to a catheter assembly 60. FIGS. 2A-2B illustrate an example catheter system 61, according to some embodiments. In some embodiments, the catheter assembly 60 may include a catheter adapter 62, which may include a distal end 64, a proximal end 66, and a lumen 68 extending through the distal end 64 of the catheter adapter 62 and the proximal end 66 of the catheter adapter 62. In some embodiments, coupling the guidewire delivery device 10 to the catheter assembly 60 may include coupling the proximal end 16 of the housing 12 to the proximal end 66 of the catheter adapter 62, as illustrated, for example, in FIG. 2A. In some embodiments, the catheter assembly 60 may include a catheter 70 extending distally from the distal end 64 of the catheter adapter 62. In some embodiments, the catheter assembly 60 may include a septum 72 configured to seal a fluid pathway through the catheter assembly 60.

In some embodiments, the tab 22 may be advanced distally within the slot 20. In some embodiments, in response to advancing the tab 22 distally, the penetration cannula 24 may penetrate the septum 72. In some embodiments, after advancing the tab 22 distally with respect to the slot 20 and the penetration cannula 24 penetrating the septum 72, the guidewire 34 may be advanced distally through the penetration cannula 24 and/or distal to the catheter 70. In some embodiments, the guidewire 34 may be advanced distally a length of the catheter 70.

In some embodiments, the septum 72 may be disposed within the lumen 68 of the catheter adapter 62. In some embodiments, coupling the guidewire delivery device 10 to the catheter assembly 60 may include coupling the proximal end 16 of the housing 12 to the proximal end 66 of the catheter adapter 62.

As illustrated, for example, in FIG. 2B, the catheter assembly 60 may include a wedge 74 disposed within the lumen 68 of the catheter adapter 62. In some embodiments, the catheter 70 may be secured within the distal end 64 of the catheter adapter 62 by the wedge 74. In some embodiments, in response to advancing the tab 22 distally with respect to the slot 20, a distal end 78 of the penetration cannula 24 may be disposed within the wedge 74.

In some embodiments, the adapter 26 may include the distal port 28, the first proximal port 30, and the second proximal port 32. In some embodiments, a needleless connector 86 may be coupled to the first proximal port 30. In some embodiments, the blood collection device 88 may be coupled to the second proximal port 32. In some embodiments, after advancing the tab 22 distally with respect to the slot 20, the guidewire 34 may be advanced distally through the first proximal port 30.

In some embodiments, after advancing the tab 22 distally with respect the slot 20, the guidewire 34 may be advanced distally through the penetration cannula 24 and distal to a distal end of the catheter 70. In some embodiments, advancing the guidewire 34 distally through the penetration cannula 24 and distal to the distal end of the catheter 70 may facilitate removal of thrombus accumulated on the distal end of the catheter 70 prior to blood collection. In some embodiments, the penetration cannula 24 may prevent damage to the septum 72 by the guidewire 34.

In some embodiments, after thrombus removal at the distal end of the catheter 70, the guidewire 34 may be retracted proximally such that a distal end 90 of the guidewire 34 is disposed within the penetration cannula 24. In some embodiments, after retracting the guidewire 34 proximally such that the distal end 90 of the guidewire 34 is disposed within the penetration cannula 24, blood may be collected into the blood collection device 88, which may be coupled to the adapter 26. In some embodiments, the blood collection device 88 may be coupled to the adapter 26 after the guidewire 34 is retracted proximally.

In some embodiments, catheter 70 may be indwelling, and the guidewire delivery device 10 may facilitate blood collection or draw from the catheter 70 that is indwelling. In some embodiments, the guidewire 34 may facilitate small gauge blood draw with a limited impacted on blood draw efficiency. In some embodiments, drawing blood through the guidewire delivery device may reduce a risk of hemolysis. In some embodiments, after blood collection, the tab 22 may be retracted proximally with respect to the slot 20. In some embodiments, in response to retracting the tab 22 proximally with respect to the slot 20, the penetration cannula 24 may be withdrawn proximally through the septum 58.

Referring now to FIGS. 2A-2C, in some embodiments, the guidewire 34 may be coiled, which may reduce thrombosis at the distal end of the catheter 70 and/or within the catheter assembly 60. In some embodiments, the guidewire 34 that is coiled may also reduce a risk of collapse of the catheter 70. In some embodiments, blood may flow through the catheter 70 into the blood collection device 88 around and/or through the guidewire 34. In some embodiments, an entirety of a length of the guidewire 34 may be coiled, from the distal end 90 to a body 92 from which the other tab 54 may extend. In some embodiments, at least the distal end 90 of the body 92 may be coiled and/or a proximal end of the guidewire 34 may be straight. In some embodiments, coils of the guidewire 34 may be spaced apart, which may facilitate blood flow through the guidewire 34. In some embodiments, all or a portion of the guidewire 34 may be constructed of metal or another suitable material.

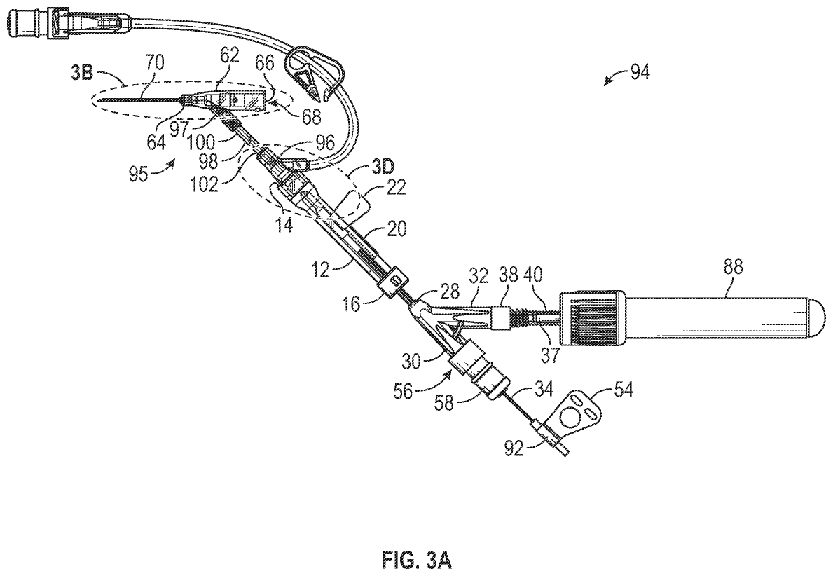

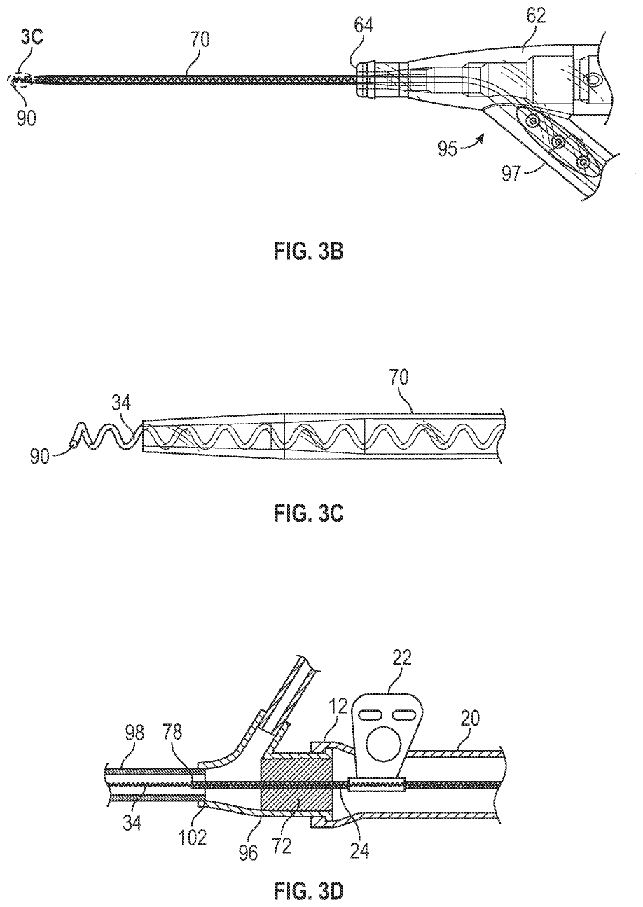

Referring now to FIGS. 3A-3D, a catheter system 94 that includes a catheter assembly 95 is illustrated, according to some embodiments. In some embodiments, the catheter system 94 may be similar or identical to the catheter system 61 of FIGS. 2A-2C in terms of one or more components and/or operation. In some embodiments, the catheter adapter 62 may include the side port 97, which may be disposed between the distal end 64 of the catheter adapter 62 and the proximal end 66 of the catheter adapter 62. In some embodiments, the catheter assembly 95 may include another adapter 96. In some embodiments, the catheter assembly 95 may include an extension tube 98, which may include a distal end 100 coupled to the side port 97 and a proximal end 102 coupled to the other adapter 96. In some embodiments, the septum 72 may be disposed within the other adapter 96. In some embodiments, coupling the guidewire delivery device 10 to the catheter assembly 60 may include coupling the proximal end 16 of the housing 12 to the other adapter 96.

In some embodiments, after the tab 22 is advanced distally with respect to the slot 20, the guidewire 34 may be advanced distally through the penetration cannula 24 and distal to the catheter 70. Additionally, in some embodiments, after the tab 22 is advanced distally with respect to the slot 20, the guidewire 34 may be rotated by the clinician.

In some embodiments, the guidewire 34 may be coiled. In some embodiments, an entirety of a length of the guidewire 34 may be coiled, from the distal end 90 to a body 92 from which the other tab 54 may extend. In some embodiments, at least the distal end 90 of the body 92 may be coiled and/or a proximal end of the guidewire 34 may be straight. In some embodiments, coils of the guidewire 34 may be spaced apart, which may facilitate blood flow through the guidewire 34.

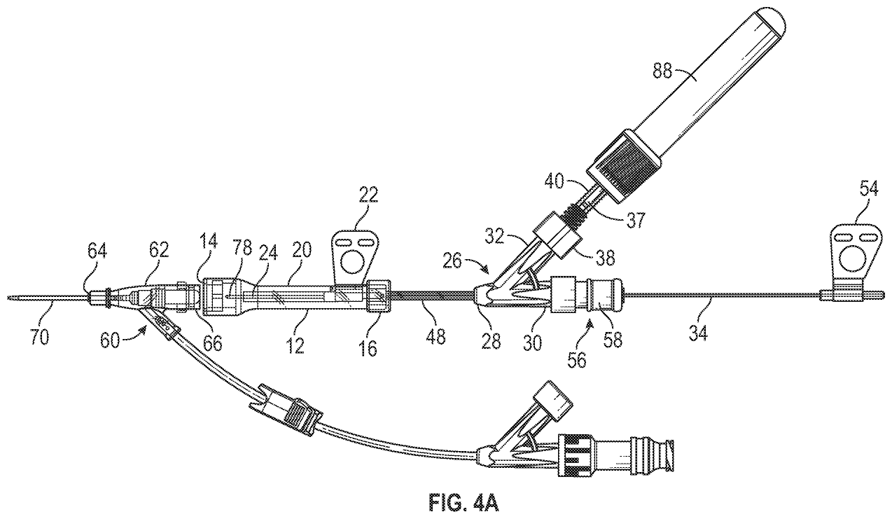

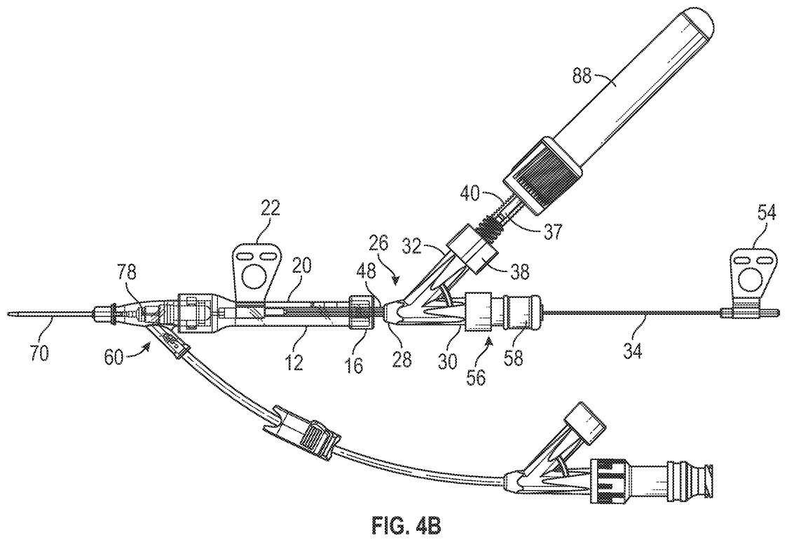

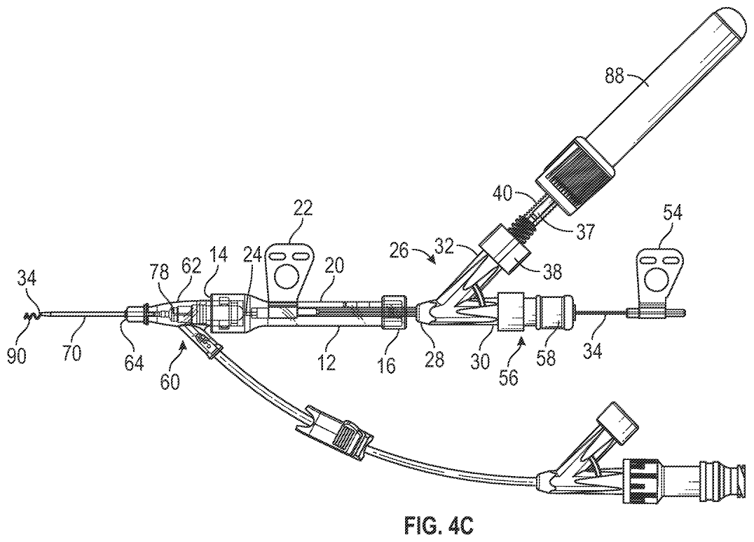

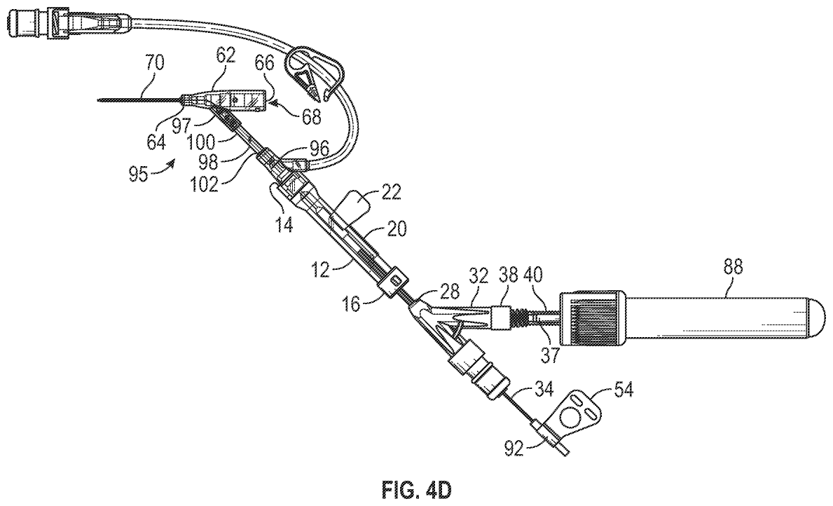

Referring now to FIG. 4A, the catheter assembly 60 is illustrated ready for insertion into vasculature of the patient, prior to coupling of the guidewire delivery device 10 to the catheter assembly 60. Referring now to FIG. 4B, the tab 22 is illustrated in the advanced position, according to some embodiments. Referring now to FIG. 4C, the guidewire 34 is illustrated in the advanced position and the catheter system 61 is ready for blood collection. Referring now to FIG. 4D, the guidewire 34 is illustrated in the advanced position and the catheter system 94 is ready for blood collection. In some embodiments, the catheter assembly 60 may be replaced with the catheter assembly 95 in FIGS. 4A-4C, and the guidewire delivery device 10 may be coupled to the other adapter 96.

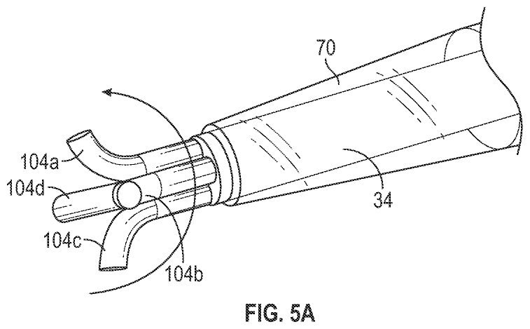

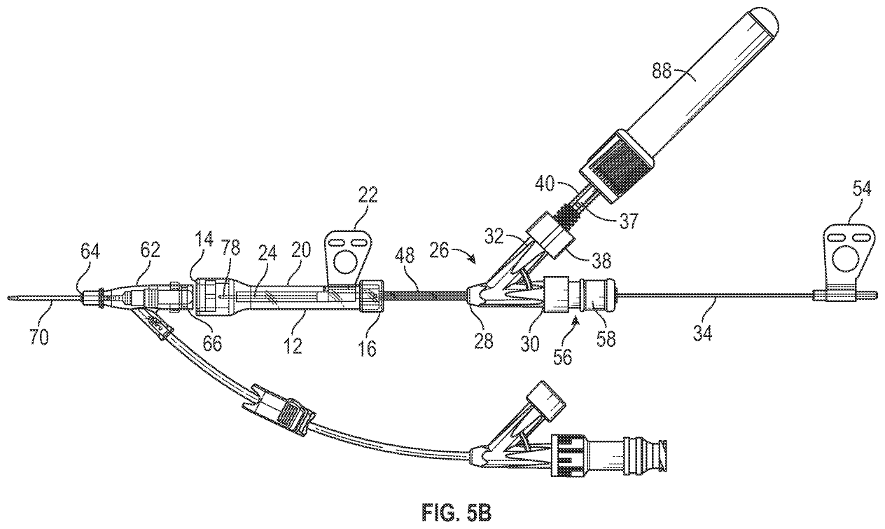

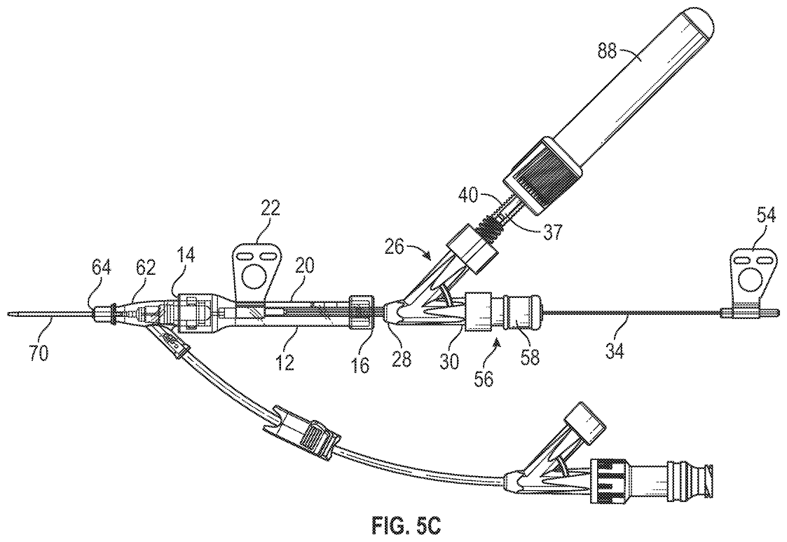

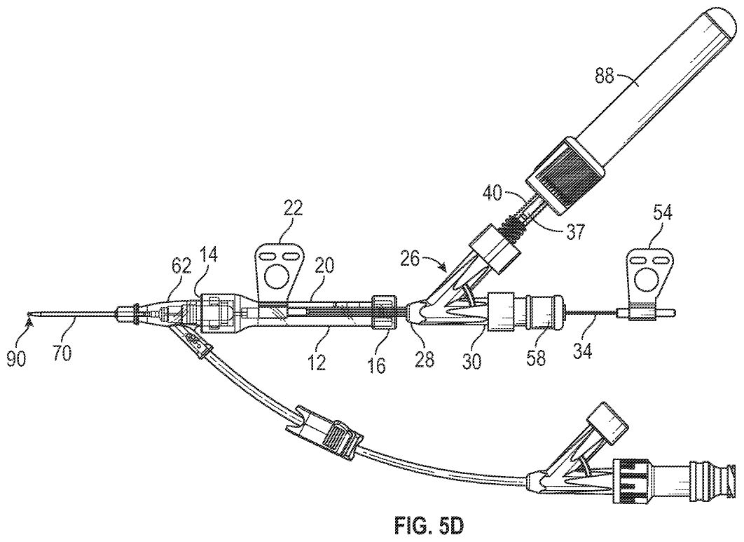

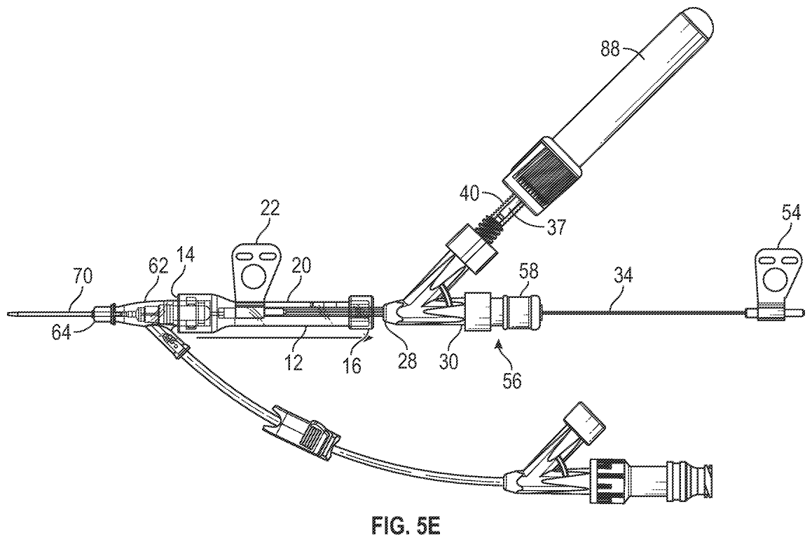

Referring now to FIG. 5A, in some embodiments, the distal end 90 of the guidewire 34 may include multiple elongated arms 104. In some embodiments, the elongated arms 104 may be configured to separate in response to removal of an inward biasing force. In some embodiments, the elongated arms 104 may be biased together in response to the elongated arms 104 being in the catheter 70. In further detail, in some embodiments, the elongated arms 104 may be touching each other or close together due to contact with an inner surface of the catheter 70. In some embodiments, the elongated arms 104 may be configured to separate or move apart from each other in response to the elongated arms 104 moving distal to the catheter 70.

In some embodiments, the elongated arms 104 may extend from a distal end of a generally cylindrical portion of the guidewire 34. In some embodiments, the generally cylindrical portion of the guidewire 34 may be monolithically formed with the elongated arms 104 as a single unit. In some embodiments, the elongated arms 104 may be constructed of a shape memory material configured to recover its original shape from a deformation when a force is applied. In some embodiments, the elongated arms 104 may be constructed of memory metal or metal.

In some embodiments, after the tab 22 is advanced distally with respect to the slot 20, the guidewire 34 may be advanced distally through the penetration cannula 24 and distal to the catheter 70. Additionally, in some embodiments, after the tab 22 is advanced distally with respect to the slot 20, the guidewire 34 may be rotated by the clinician. In some embodiments, the guidewire 34 may be used with the catheter system 61 and/or the catheter system 94.

Referring now to FIGS. 5B-5E, the guidewire 34 is illustrated in various positions to facilitate blood collection. FIG. 5E illustrates the guidewire 34 in a retracted position after blood collection in the blood collection device 88, according to some embodiments.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

We claim:

1. A guidewire delivery device, comprising:
a housing, comprising a distal end, a proximal end, a lumen extending through the distal end of the housing and the proximal end of the housing, and a slot disposed between the distal end of the housing and the proximal end of the housing;
a tab moveable with respect to the slot;
a penetration cannula coupled to the tab and extending in a distal direction; and
an adapter proximal to and in fluid communication with the penetration cannula.

2. The guidewire delivery device of claim 1, wherein the penetration cannula and the adapter are configured to receive a coiled guidewire therethrough.

3. The guidewire delivery device of claim 1, further comprising a lumen extending from a proximal end of the penetration cannula to the adapter.

4. The guidewire delivery device of claim 3, wherein the lumen extends through the proximal end of the housing, wherein in response to movement of the tab with respect to the slot in a distal direction, the adapter moves closer to the proximal end of the housing.

5. The guidewire delivery device of claim 3, further comprising a body and an extension tube extending proximally from the body to the adapter, wherein the tab extends from the body, wherein the penetration cannula extends distally from the body.

6. The guidewire delivery device of claim 1, wherein the adapter comprises a Y-adapter.

7. The guidewire delivery device of claim 1, wherein the distal end of the housing comprises a luer adapter configured to couple to a side port or a distal end of a catheter adapter.

8. The guidewire delivery device of claim 1, wherein the penetration cannula is 20 G.

9. The guidewire delivery device of claim 1, further comprising a guidewire extending distally through the adapter.

10. The guidewire delivery device of claim 9, wherein the guidewire is coiled.

11. The guidewire delivery device of claim 9, wherein a distal end of the guidewire comprises a plurality of elongated arms, wherein the elongated arms are configured to separate in response to removal of an inward biasing force.

12. A method of blood collection, comprising:
coupling a guidewire delivery device to a catheter assembly, wherein the guidewire delivery device comprises:
a housing, comprising a distal end, a proximal end, a lumen extending through the distal end of the housing and the proximal end of the housing, and a slot disposed between the distal end of the housing and the proximal end of the housing;
a tab moveable with respect to the slot;
a penetration cannula coupled to the tab and extending in a distal direction; and
an adapter proximal to and in fluid communication with the penetration cannula;
wherein the catheter assembly comprises:
a catheter adapter, comprising a distal end, a proximal end, and a lumen extending through the distal end of the catheter adapter and the proximal end of the catheter adapter;
a catheter extending distally from the distal end of the catheter adapter; and
a septum configured to seal a fluid pathway through the catheter assembly;
advancing the tab distally within the slot, wherein in response to advancing the tab distally, the penetration cannula penetrates the septum; and
after advancing the tab distally with respect to the slot, advancing the guidewire distally through the penetration cannula and distal to the catheter.

13. The method of claim 12, wherein the septum is disposed within the lumen of the catheter adapter, wherein coupling the guidewire delivery device to the catheter assembly further comprising coupling the proximal end of the housing to the proximal end of the catheter adapter.

14. The method of claim 12, wherein the catheter assembly further comprises:
a side port disposed between the distal end of the catheter adapter and the proximal end of the catheter adapter;
another adapter; and
an extension tube, comprising a distal end coupled to the side port and a proximal end coupled to the other adapter,
wherein the septum is disposed within the other adapter, wherein coupling the guidewire delivery device to the catheter assembly further comprises coupling the proximal end of the housing to the other adapter.

15. The method of claim 12, wherein the catheter assembly further comprises a wedge disposed within the lumen of the catheter adapter, wherein the catheter is secured within the distal end of the catheter adapter by the wedge, wherein in response to advancing the tab distally with respect to the slot, a distal end of the penetration cannula is disposed within the wedge.

16. The method of claim 12, wherein the guidewire is coiled.

17. The method of claim 12, wherein a distal end of the guidewire comprises a plurality of elongated arms, wherein the elongated arms are biased together in response to the elongated arms being in the catheter, wherein the elongated arms are configured to separate in response to the elongated arms moving distal to the catheter, wherein after the tab is advanced distally with respect to the slot, the guidewire is advanced distally through the penetration cannula and distal to the catheter and rotated.

18. The method of claim 12, wherein the adapter comprises a distal port, a first proximal port, and a second proximal port, wherein a needleless connector is coupled to the first proximal port, wherein a blood collection device is coupled to the second proximal port, wherein after advancing the tab distally with respect to the slot, the guidewire is advanced distally through the first proximal port.

19. The method of claim 12, wherein after advancing the tab distally with respect to the slot, the guidewire is advanced distally through the penetration cannula and distal to the catheter.

20. The method of claim 12, further comprising:
retracting the guidewire proximally such that a distal end of the guidewire is disposed within the penetration cannula; and
after retracting the guidewire proximally such that the distal end of the guidewire is disposed within the penetration cannula, collecting blood through a blood collection device coupled to the adapter.
